(19) United States
(12) Patent Application Publication
(10) Pub. No.: US 2021/0380519 A1
(43) Pub. Date: Dec. 9, 2021

(54) PROCESS FOR PRE-HEATING BOILER FEED WATER IN THE MANUFACTURE OF PURIFIED AROMATIC CARBOXYLIC ACIDS

(71) Applicant: BP CORPORATION NORTH AMERICA INC., Houston, TX (US)

(72) Inventor: Frederick CLARK, Naperville, IL (US)

(21) Appl. No.: 16/635,804

(22) PCT Filed: Aug. 1, 2018
(86) PCT No.: PCT/US2018/044739

§ 371 Date: Jan. 31, 2020

Related U.S. Application Data
(60) Provisional application No. 62/539,631, filed on Aug. 1, 2017.

(51) Int. Cl. C07C 51/44

(52) U.S. Cl. CPC C07C 51/445

(57) ABSTRACT

Processes for manufacturing purified aromatic carboxylic acids include: generating high-pressure steam from boiler feed water supplied to a boiler, the boiler producing a flue gas; removing a portion of the flue gas from the boiler and pre-heating the boiler feed water with removed flue gas and/or pre heating at least a portion of the boiler feed water prior to its introduction into the boiler with a first portion of the high-pressure steam; heating a crude aromatic carboxylic acid in a heating zone using the high-pressure steam, whereby the high pressure steam is condensed in the heating zone to form a high-pressure condensate; and purifying the crude aromatic carboxylic acid to form a purified aromatic carboxylic acid; wherein the boiler feed water comprises at least a portion of the high-pressure condensate.

### TECHNICAL FIELD

The present teachings relate generally to processes for manufacturing purified aromatic carboxylic acids, and in particular, to processes for pre-heating boiler feed water.

### BACKGROUND

Terephthalic acid (TA) and other aromatic carboxylic acids may be used in the manufacture of polyesters (e.g., via their reaction with ethylene glycol and/or higher alkylene glycols). Polyesters in turn may be used to make fibers, films, containers, bottles, other packaging materials, molded articles, and the like.

In commercial practice, aromatic carboxylic acids have been made by liquid phase oxidation of methyl-substituted benzene and naphthalene feedstocks in an aqueous acetic acid solvent. The positions of the methyl substituents correspond to the positions of carboxyl groups in the aromatic carboxylic acid product. Air or other sources of oxygen (e.g., typically in a gaseous state) have been used as oxidants in the presence, for example, of a bromine-promoted catalyst that contains cobalt and manganese. The oxidation is exothermic and yields aromatic carboxylic acid together with by-products, including partial or intermediate oxidation products of the aromatic feedstock, and acetic acid reaction products (e.g., methanol, methyl acetate, and methyl bromide). Water is also generated as a by-product.

Pure forms of aromatic carboxylic acids are oftentimes desirable for the manufacture of polyesters to be used in important applications (e.g., fibers and bottles). Impurities in the acids (e.g., by-products generated from oxidation of aromatic feedstocks and, more generally, various carbonyl-substituted aromatic species) are thought to cause and/or correlate with color formation in polyesters made therefrom, which in turn leads to off-color in polyester converted products. Aromatic carboxylic acids having reduced levels of impurities may be made by further oxidizing crude products from liquid phase oxidation as described above at one or more progressively lower temperatures and oxygen levels. In addition, partial oxidation products may be recovered during crystallization and converted into the desired acid product.

Pure forms of terephthalic acid and other aromatic carboxylic acids having reduced amounts of impurities—for example, purified terephthalic acid (PTA)—have been made by catalytically hydrogenating less pure forms of the acids or so-called medium purity products in solution at elevated temperature and pressure using a noble metal catalyst. Less pure forms of the acids may include crude product that contains aromatic carboxylic acid and by-products from liquid phase oxidation of the aromatic feedstock. In commercial practice, liquid phase oxidation of alkyl aromatic feed materials to crude aromatic carboxylic acid, and purification of the crude product, are oftentimes conducted in continuous integrated processes in which crude product from the liquid phase oxidation is used as a starting material for the purification.

Purification of crude aromatic carboxylic acid has been accomplished through hydrogenation. Crude aromatic carboxylic acid is usually pre-heated prior to being fed to the hydrogenation reactor, which typically operates at a temperature of about 260° C. to about 290° C. One manner in which such pre-heating is accomplished is through indirect heat exchange with high pressure steam. The high pressure steam is condensed during heat exchange, and the resulting condensate may be let down to form low pressure condensate and low pressure steam which may be used in other process steps. In the alternative, the high pressure condensate may be recycled as feed water to the boiler used to generate steam.

The fuel costs associated with generation of the high pressure steam contributes to the overall variable costs of the process for manufacturing the purified aromatic carboxylic acid. Furthermore, the formation NOx in the flue gas of the steam boiler often requires costly remediation. There continues to be a desire to reduce such variable costs through more efficient energy management and pollution control strategies.

### SUMMARY

According to one aspect of the invention, a process for manufacturing a purified carboxylic acid comprises generating high-pressure steam from boiler feed water supplied to a boiler, the boiler producing a flue gas; removing a portion of the flue gas from the boiler and pre-heating the boiler feed water with removed flue gas; heating a crude aromatic carboxylic acid in a heating zone using the high-pressure steam, whereby the high pressure steam is condensed in the heating zone to form a high-pressure condensate; and purifying the crude aromatic carboxylic acid to form a purified aromatic carboxylic acid; wherein the boiler feed water comprises at least a portion of the high-pressure condensate.

A process for manufacturing a purified aromatic carboxylic acid comprises generating high-pressure steam from boiler feed water supplied to a boiler; pre-heating at least a portion of the boiler feed water prior to its introduction into the boiler with a first portion of the high-pressure steam; heating a crude aromatic carboxylic acid in a heating zone with a second portion of the high-pressure steam, whereby the high pressure steam is condensed in the heating zone to form a high-pressure condensate; and purifying the crude aromatic carboxylic acid to form a purified aromatic carboxylic acid; wherein the boiler feed water comprises at least a portion of the high-pressure condensate.

Other aspects of the present invention will be apparent in view of the description that follows.

### BRIEF DESCRIPTION OF THE DRAWINGS

FIG. 1 shows a process flow diagram for the manufacture of purified forms of aromatic carboxylic acids in accordance with one embodiment of the present invention.

### DETAILED DESCRIPTION

By way of general introduction, the present invention is directed to processes for manufacturing purified aromatic carboxylic acids using efficient heat exchange configurations in the pre-heating of crude aromatic carboxylic acids prior to purification. High-pressure steam is used to heat crude aromatic carboxylic acid in a pre-heating zone prior to the purification of the crude aromatic carboxylic acid. At least a portion of high-pressure condensate generated from the condensation of the high pressure steam in the pre-heating zone may be recycled to provide at least a portion of boiler feed water from which the high pressure steam is generated. The boiler water feed is pre-heated with a portion of the boiler flue gas removed from the boiler and/or with a first portion of the high-pressure steam.

Additional features of the above-described processes for manufacturing purified forms of aromatic carboxylic acid in accordance with the present teachings will now be described in reference to the drawing figures.

Processes for manufacturing purified aromatic carboxylic acids from substituted aromatic hydrocarbons, along with ancillary processes for recovering energy and purifying waste streams are generally known in the art and more fully described, for example, in U.S. Pat. Nos. 5,723,656, 6,137,001, 7,935,844, 7,935,845, 8,173; 834, and 9,315,441.

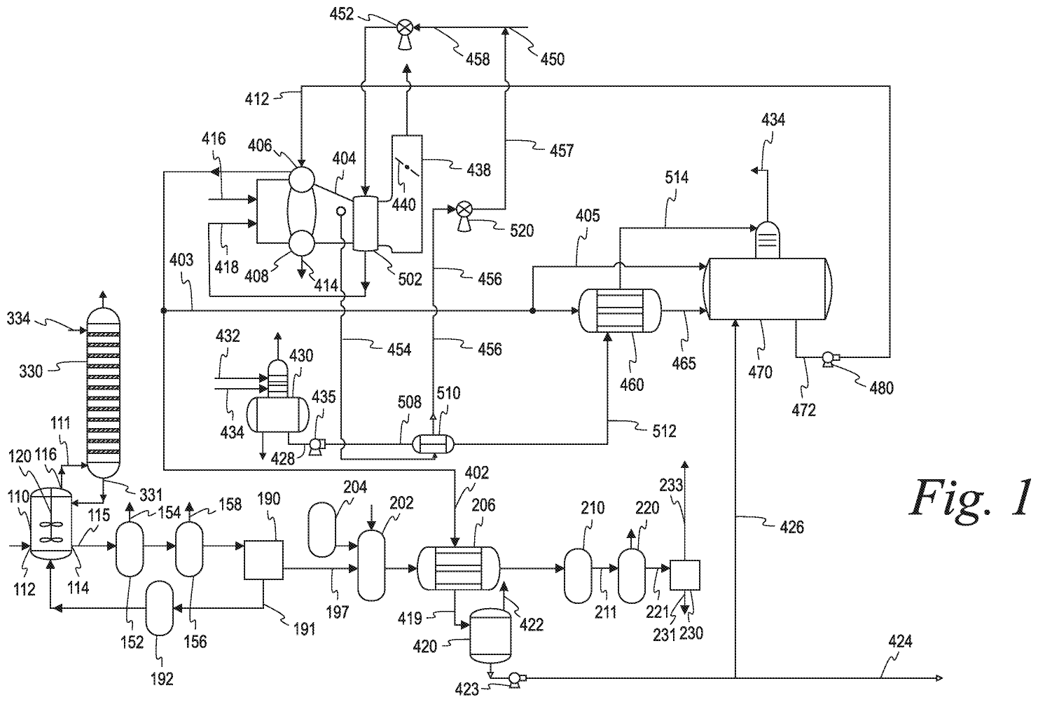

FIG. 1 shows a simplified process flow diagram for manufacturing purified forms of aromatic carboxylic acids in accordance with the present invention. Liquid and gaseous streams and materials used in the process represented in FIG. 1 may be directed and transferred through suitable transfer lines, conduits, and piping constructed, for example, from materials appropriate for process use and safety. It will be understood that particular elements may be physically juxtaposed and, where appropriate, may have flexible regions, rigid regions, or a combination of both. In directing streams or compounds, intervening apparatuses and/or optional treatments may be included. By way of example, pumps, valves, manifolds, gas and liquid flow meters and distributors, sampling and sensing devices, and other equipment (e.g., for monitoring, controlling, adjusting, and/or diverting pressures, flows and other operating parameters) may be present.

In a representative embodiment, such as may be implemented as shown in FIG. 1, liquid feed material comprising, by way of example, at least about 99 wt. % of a substituted aromatic hydrocarbon feed material, a monocarboxylic acid solvent, an oxidation catalyst, a catalyst promoter, and air are continuously charged to oxidation reaction vessel 110 through inlets, such as inlet 112. In some embodiments, vessel 110 is a pressure-rated, continuous-stirred tank reactor.

In some embodiments, stirring may be provided by rotation of an agitator 120, the shaft of which is driven by an external power source (not shown). Impellers mounted on the shaft and located within the liquid body are configured to provide forces for mixing liquids and dispersing gases within the liquid body, thereby avoiding settling of solids in the lower regions of the liquid body.

Suitable aromatic feed materials for the oxidation generally comprise an aromatic hydrocarbon substituted at one or more positions, normally corresponding to the positions of the carboxylic acid groups of the aromatic carboxylic acid being prepared, with at least one group that is oxidizable to a carboxylic acid group. The oxidizable substituent or substituents can be alkyl groups, such as a methyl, ethyl or isopropyl groups, or groups already containing oxygen, such as a hydroxyalkyl, formyl or keto group. The substituents can be the same or different. The aromatic portion of feedstock compounds can be a benzene nucleus or it can be bi- or polycyclic, such as a naphthalene nucleus. Examples of useful feed compounds, which can be used alone or in combinations, include toluene, ethylbenzene and other alkyl-substituted benzenes, o-xylene, p-xylene, m-xylene, tolualdehydes, toluic acids, alkyl benzyl alcohols, 1-formyl-4-methylbenzene, 1-hydroxymethyl-4-methylben-zene, methylacetophenone, 1,2,4-trimethylbenzene, 1-formyl-2,4-dimethyl-benzene, 1,2,4,5-tetramethyl-benzene, alkyl-, formyl-, acyl-, and hydroxylmethyl-substituted naphthalenes, such as 2,6-diethylnaphthalene, 2,6-diethylnaphalene, 2,7-dimethylnaphthalene, 2,7-diethylnaphthalene, 2-formyl-6-methylnaphthalene, 2-acyl-6-methylnaphthalene, 2-methyl-6-ethylnaphthalene and partially oxidized derivatives of the foregoing.

For manufacture of aromatic carboxylic acids by oxidation of their correspondingly substituted aromatic hydrocarbon pre-cursors, e.g., manufacture of benzoic acid from mono-substituted benzenes, terephthalic acid from para-disubstituted benzenes, phthalic acid from ortho-disubstituted benzenes, and 2,6 or 2,7 naphthalene dicarboxylic acids from, respectively, 2,6- and 2,7-disubstituted naphthalenes, it is preferred to use relatively pure feed materials, and more preferably, feed materials in which content of the pre-cursor corresponding to the desired acid is at least about 95 wt. %, and more preferably at least 98 wt. % or even higher. In one embodiment, the aromatic hydrocarbon feed for use to manufacture terephthalic acid comprises para-xylene.

Solvent for the liquid phase reaction of aromatic feed material to aromatic carboxylic acid product in the liquid phase oxidation step comprises a low molecular weight monocarboxylic acid, which is preferably a C₁-C₅ monocarboxylic acid, for example acetic acid, propionic acid, butyric acid, valeric acid and benzoic acid.

Catalysts used for the liquid oxidation comprise materials that are effective to catalyze oxidation of the aromatic feed material to aromatic carboxylic acid. Preferred catalysts are soluble in the liquid phase reaction mixture used for oxidation because soluble catalysts promote contact among catalyst, oxygen gas and liquid feed materials; however, heterogeneous catalyst or catalyst components may also be used. Typically, the catalyst comprises at least one heavy metal component. Examples of suitable heavy metals include cobalt, manganese, vanadium, molybdenum; chromium, iron, nickel, zirconium, cerium or a lanthanide metal such as hafnium. Suitable forms of these metals include, for example, acetates, hydroxides, and carbonates. Preferred catalysts comprise cobalt, manganese, combinations thereof and combinations with one or more other metals and particularly hafnium, cerium and zirconium.

In preferred embodiments, catalyst compositions for liquid phase oxidation also comprise a promoter, which promotes oxidation activity of the catalyst metal, preferably without generation of undesirable types or levels of by-products. Promoters that are soluble in the liquid reaction mixture used in oxidation pre preferred for promoting contact among catalyst, promoter and reactants. Halogen compounds are commonly used as a promoter, for example hydrogen halides, sodium halides, potassium halides, ammonium halides, halogen-substituted hydrocarbons, halogen-substituted carboxylic acids and other halogenated compounds. Preferred promoters comprise at least one bromine source. Suitable bromine sources include bromo-anthracenes, Br₂, HBr, NaBr, KBr, NH₄Br, benzyl-bromide, bromo acetic acid, dibromo acetic acid, tetrabromoethane, ethylene dibromide, bromoacetyl bromide and combinations thereof. Other suitable promoters include aldehydes and ketones such as acetaldehyde and methyl ethyl ketone. Reactants for the liquid phase reaction of the oxidation step also include a gas comprising molecular oxygen. Air is conveniently used as a source of oxygen gas. Oxygen-enriched air, pure oxygen and other gaseous mixtures comprising molecular oxygen, typically at levels of at least about 10 vol. %, also are useful.

The substituted aromatic hydrocarbon is oxidized in reactor 110, to form a crude aromatic carboxylic acid and by-products. In one embodiment, for example, paraxylene is converted to terephthalic acid and by-products that may form in addition to terephthalic acid include partial and intermediate oxidation products (e.g., 4-carboxybenzaldehyde, 1,4-hydroxymethyl benzoic acid, p-toluic acid, benzoic acid, and the like, and combinations thereof). Since the oxidation reaction is exothermic, heat generated by the reaction may cause boiling of the liquid phase reaction mixture and formation of an overhead vapor phase that comprises vaporized acetic acid, water vapor, gaseous by-products from the oxidation reaction, carbon oxides, nitrogen from the air charged to the reaction, unreacted oxygen, and the like, and combinations thereof.

The overhead vapor is removed from the reactor 110 through vent 116 and sent in a stream 111 to a separation zone, which in the embodiment shown is high-pressure distillation column 330. The separation zone is configured to separate water from the solvent monocarboxylic acid and return a solvent-rich liquid phase to the reactor via line 331. A water rich gas phase is removed from the separation zone via line 332 and is further processed in off-gas treatment zone 350. Reflux 334 is returned to the column 330. Examples of further processing of the overhead gas stream and reflux options for the column 330 are more fully described in U.S. Pat. Nos. 5,723,656, 6,137,001, 7,935,844, 7,935,845, and 8,173,834. Liquid effluent comprising solid crude aromatic carboxylic acid product is slurried in the liquid phase reaction mixture is removed from reaction vessel 110 through slurry outlet 114 and directed in stream 115 to a crystallization zone for recovery of a solid product.

In the embodiment of the invention illustrated in FIG. 1, the crystallization zone includes multiple stirred crystallization vessels, 152 and 156 in series and in flow communication for transfer of product slurry from vessel 152 to vessel 156. Cooling in the crystallization vessels is accomplished by pressure release, with the slurry cooled in vessel 152 to a temperature in the range of about 150-190° C. and then further to about 110-150° C. in vessel 156. One or more of the crystallization vessels is vented, as at 154 and 158, respectively, for removal to heat exchange means (not shown) of vapor resulting from pressure let down and generation of steam from the flashed vapor. Vapor removed from one or more upstream crystallization vessels, such as vessel 152, to heat exchange means is preferably condensed and liquid condensate comprising water, acetic acid solvent and soluble products and by-products of the oxidation can directed to one or more downstream crystallization vessels, as at 156, to allow for recovery of crystallizable components such as crude aromatic carboxylic acid and oxidation by-products entering and condensed from the flashed vapors from one or more upstream vessel.

Crystallization vessel 156 is in fluid communication with a solid-liquid separation device 190, which is adapted to receive from the crystallization vessel a slurry of solid product comprising the crude aromatic carboxylic acid and oxidation by-products in a mother liquor from the oxidation comprising monocarboxylic acid solvent and water, and to separate a crude solid product comprising terephthalic acid and by-products from the liquid. Separation device 190 is a centrifuge, rotary vacuum filter or pressure filter. In preferred embodiments of the invention, the separation device is a pressure filter adapted for solvent exchange by positive displacement under pressure of mother liquor in a filter cake with wash liquid comprising water. The oxidation mother liquor that results from the separation exits separation device 190 in stream 191 for transfer to mother liquor drum 192. A major portion of the mother liquor is transferred from drum 192 to oxidation reactor 110 for return to the liquid phase oxidation reaction of acetic acid, water, catalyst and oxidation reaction by-products dissolved or present as fine solid particles in the mother liquor. Crude solid product and impurities comprising oxidation by-products of the feedstock is conveyed, with or without intermediate drying and storage, from separation device 190 to purification solution make up vessel 202 in stream 197. The crude solid product is slurried in make up vessel 202 in purification reaction solvent, all or at least a portion, and preferably about 60 to about 100 wt. %, of which, comprises a second liquid phase from an off-gas separation of water and acetic acid in a vapor phase removed from reactor 110 to column 330 and by-products of the oxidation. If used, make up solvent, such as fresh demineralized water or suitable recycle streams such as liquid condensed from vapors resulting from pressure letdown in crystallization of purified terephthalic acid product as discussed below, can be directed to make up tank 202 from vessel 204. Slurry temperature in the make up tank preferably is about 80 to about 100° C.

Crude aromatic carboxylic acid product is dissolved to form a purification reaction solution by heating, for example to about 260 to about 290° C. in makeup tank 202 and by passage through a heating zone comprising one or more heat exchangers 206 as it is transferred to purification reactor 210. In reactor 210, the purification reaction solution is contacted with hydrogen under pressure preferably ranging from about 85 to about 95 bar (g) in the presence of a hydrogenation catalyst.

Catalysts suitable for use in purification hydrogenation reactions comprise one or more metals having catalytic activity for hydrogenation of impurities in impure aromatic carboxylic acid products, such as oxidation intermediates and by-products and/or aromatic carbonyl species. The catalyst metal preferably is supported or carried on a support material that is insoluble in water and unreactive with aromatic carboxylic acids under purification process conditions. Suitable catalyst metals are the Group VIII metals of the Periodic Table of Elements (IUPAC version), including palladium, platinum, rhodium, osmium, ruthenium, iridium, and combinations thereof. Palladium or combinations of such metals that include palladium are most preferred. Carbons and charcoals with surface areas of several hundreds or thousands m²/g surface area and sufficient strength and attrition resistance for prolonged use under operating conditions are preferred supports, Metal loadings are not critical but practically preferred loadings are about 0.1 wt % to about 5 wt % based on total weight of the support and catalyst metal or metals. Preferred catalysts for conversion of impurities present in impure aromatic carboxylic acid products contain about 0.1 to about 3 wt % and more preferably about 0.2 to about 1 wt % hydrogenation metal. In one particular embodiment, the metal comprises palladium.

A portion of the purification liquid reaction mixture is continuously removed from hydrogenation reactor 210 in stream 211 to crystallization vessel 220 where purified aromatic carboxylic acid product and reduced levels of impurities are crystallized from the reaction mixture by reducing pressure on the liquid. The resulting slurry of purified aromatic carboxylic acid and liquid formed in vessel 220 is directed to solid-liquid separation apparatus 230 in stream line 221. Vapors resulting from pressure letdown in the crystallization can be condensed by passage to heat exchangers (not shown) for cooling and the resulting condensed liquid redirected to the process, for example as recycle to purification feed makeup tank 202, through suitable transfer lines (not shown). Purified aromatic carboxylic acid product exits solid-liquid separation device 230 in stream 231. The solid-liquid separation device can be a centrifuge, rotary vacuum filter, a pressure filter or combinations of one or more thereof.

Purification mother liquor from which the solid purified aromatic carboxylic acid product is separated in solid-liquid separator 230 comprises water, minor amounts of dissolved and suspended aromatic carboxylic acid product and impurities including hydrogenated oxidation by-products dissolved or suspended in the mother liquor. Purification mother liquor is directed in stream 233 may be sent to waste water treatment facilities or alternatively may be used a reflux 334 to the column 330, as more fully described, for example, in U.S. Pat. Nos. 5,723,656, 6,137,001, 7,935,844, 7,935,845, and 8,173,834.

As discussed above, crude aromatic carboxylic acid product is heated in a heating zone having heat exchanger 206. Those skilled in the art appreciate that although one heat exchanger is shown, the heating zone may include multiple heat exchangers including pre-heaters upstream of heat exchanger 206. In one embodiment, the heat exchanger is a tube and shell exchanger in which the crude aromatic carboxylic acid is heated by indirect contact heating with high pressure steam supplied by line 402.

The high pressure steam 402 is generated by a boiler 404. In one embodiment, the boiler 404 is a standard type-D Nebraska boiler available from Cleaver-Brooks of Lincoln, Nebr. The boiler 404 includes a steam drum 406 and a mud drum 408 connected by a plurality of riser and downcomer tubes 410. Boiler feed water is introduced into the steam drum 406 through line 412. The boiler feed water is delivered as a liquid at pressures slightly exceeding the pressure of the steam drum 406 and at temperatures which are sub-cooled relative to the delivery pressure. The density of the boiler feed water entering the steam drum 406 is greater compared with the density of the two-phase liquid-vapor water mixture in the steam drum 406. This density gradient thereby promotes a thermosiphon effect as the entering, higher density liquid flows downward through the downcomer tubes 410 and into the lower mud drum 408 which, in turn, forces lower density, two-phase water mixtures to flow upward in the riser tubes 410 from the mud drum 408 into the steam drum 406. High pressure steam is removed from the steam drum 406 through line 402. Bottom blowdown, comprising water with impurities, is removed from the mud drum 408 through line 414 at a rate of about 1% to 3% of the boiler feed water 412 entering the steam drum to avoid the build-up of corrosive materials. A fuel, such as natural gas, is injected through line 416 and a source of oxygen, such as air, is introduced through line 418 for providing the combustion heat source (not shown) for the boiler 404. In one embodiment, the oxygen source in line 418 is preheated in an upstream gas-to-gas air preheater 502 with hot flue gas from the boiler combustion zone. The cooled flue gas exiting preheater 502 passes into stack 438, with its flow optionally controlled with damper 440.

The boiler feed water 412 includes at least a portion of the high pressure condensate 419 that is formed by the condensation of the high pressure steam 402 in the shell side of the heat exchanger 206, In one embodiment, high pressure condensate 419 exiting the heat exchanger 206 is introduced into flash drum 420, which is preferably maintained at pressures as close as possible to condensate 419 to minimize generation of flashed steam 422, which otherwise is sent to other parts of the process (not shown). Pressures in the flash drum 420 can range between about 40 bar (g) to 90 bar (g) with associated temperatures ranging from 250° C. to 305° C. In one embodiment, pressures in the flash drum 420 can range between about 70 bar (g) to 85 bar (g) with associated temperatures ranging from 285° C. to 300° C. A portion of the high pressure condensate exiting flash drum 420 may be withdrawn through line 424 to be used in other parts of the process. However, at least a portion of the high pressure condensate exiting flash drum 420 through line 426 is further pressurized and sub-cooled by pump 423 and sent to a high pressure dearerator 470, where the condensate is mixed with makeup water 514 from at least one other source as well as high pressure steam 405 and condensate 465. Alternatively, the high pressure condensate 424 can be sent to deaerator 470 without a pump, if sufficient pressure differential exists between drum 420 and deaerator 470. Pressures in the high pressure deaerator 470 can range between about 40 bar (g) to 90 bar (g)—with associated temperatures ranging from 250° C. to 305° C., In one embodiment, pressures in the high pressure deaerator can range between 60 bar (g) to 75 bar (g)—with associated temperatures ranging from 275° C. to 290° C. Water 472 exiting the high pressure deaerator 470 is then further pressurized and subcooled by pump 480 before being recycled as boiler feed water 412. In one embodiment, at least 65 wt %, or up to at least 97 wt %, of the high pressure condensate 419 is recycled for use as boiler feed water.

In one embodiment, makeup boiler feed water is initially at lower temperatures—ranging between about 100° C. to 154° C.—compared with the high pressure condensate—with temperatures ranging between about 250° C. to 305° C.—prior to their combination. In one embodiment, makeup boiler feed water 428 is provided by a low pressure deaerator 430, which removes dissolved oxygen from deionized water 432 using vent steam 434 let down from the high pressure deaerator 470, as well as from other low pressure steam sources. Pressures in the low pressure deaerator can range between about 0 bar (g) to 3.5 bar (g)—with associated temperatures ranging between 100° C. to 150° C., The deaerated make-up boiler feed water 428 exiting the low pressure deaerator 430 is then further pressurized and sub-cooled by pump 435 to pressures ranging from about 40 bar (g) up to 120 bar (g) and sent to at least one additional preheating step prior to mixing with high pressure condensate 426 in the high pressure deaerator 470.

In one embodiment, the makeup boiler feed water 508 discharged from pump 435 is preheated in heat exchanger 510 using a portion of flue gas 454 extracted from the boiler breach, upstream of the gas-to-gas air preheater 502. In one embodiment, the portion of flue gas 454 removed from the boiler is 50% or less of the volume of flue gas produced in the boiler. In another embodiment, the portion of flue gas 454 removed from the boiler is 30% or less of the volume of flue gas produced in the boiler. In another embodiment, the portion of flue gas 454 removed from the boiler is 20% or less of the volume of flue gas produced in the boiler. In another embodiment, the portion of flue gas 454 removed from the boiler is at least 5% of the volume of flue gas produced in the boiler. The cooled flue gas 456 exiting exchanger 510 is pressurized in fan 520 and then mixed with a source of oxygen 450, such as fresh air, prior to entering the suction side of another pressurizing fan 452, which directs the flue gas and air mixture to the boiler combustion zone (not shown).

In another embodiment, the makeup boiler feed water 512 is further preheated in exchanger 460 using small flows of “sacrificial steam” 403 extracted as a portion of the primary flow of the high pressure steam 402, as shown in FIG. 1. The preheated makeup boiler feed water 514 then passes into the top tray of the high pressure deaerator 470, while condensed steam 465 exiting exchanger 460 passes into the bottom drum of deaerator 470. In addition, to maintain the desired target pressure in deaerator 470, a small portion of sacrificial steam 405 by-passes exchanger 460 and enters directly into the bottom drum of deaerator 470. Flashed steam generated inside deaerator 470 passes into the upper trayed section and removes dissolved oxygen (if any) in the downward-flowing preheated makeup boiler feed water 514. A small steam vent 434 exits the top of deaerator 470. Effluent 472 exits the bottom of deaerator 470 and comprises a mixture of sacrificial condensed steam 465 that exited exchanger 460, sacrificial steam 405 condensed directly inside deaerator 470, preheated makeup boiler feed water 514 that exited exchanger 460, and high pressure condensate 426 recycled from flash drum 420. Effluent 472 is further pressurized and subcooled in pump 480 and delivered as boiler feed water 412 directly to the boiler steam drum 406, as shown in FIG. 1.

Flue gas partially recycled and mixed with fresh air prior to entering the boiler combustion zone provides benefits of reduced thermal NOX emissions in the boiler by increasing the flow of inerts, such as nitrogen, into the combustion zone. This in turn reduces both the flame temperature and the thermal driving force to form NOX from the reaction of nitrogen with oxygen in the combustion zone. A further description of the impact of flue gas recycle on reducing NOX emissions is found in “Burners for Fired Heaters in General Refinery Service,” API Recommended Practice 535, 2nd Ed., American Petroleum Institute, January 2006, pp. 13-16.

One adverse consequence of flue gas recycle is that it can potentially reduce a boiler's rating since decreased flame temperatures in the combustion zone also reduce the thermal driving force for steam generation. However, by preheating the makeup boiler feed water in exchanger 510 with partially recycled flue gas—that otherwise would exit the stack as waste heat—the required duty in the second downstream exchanger 460 is reduced, which results in less flow of sacrificial steam 403 to this exchanger. Less flow of sacrificial steam 403 to exchanger 460 results in more boiler steam 402 for the process exchanger 206 assuming the boiler burners are fuel-limited; or, alternatively—if the flow of boiler steam 402 to process exchanger 206 is to be kept constant—reduced flow of sacrificial steam 403 to exchanger 460 results in reduced consumption of fuel 416 in the boiler for a variable cost savings. When designing exchanger 510, care should be taken to keep the flue gas temperature sufficiently elevated above its acid dew point to minimize impacts from acid corrosion of metal surfaces from any condensed vapour in the flue gas. Acid dew points generally increase with increasing sulphur content in the flue gas—which depends on the fuel type. Natural gas fuels generally have very low sulphur contents, allowing use of less expensive carbon steel metallurgy for exchanger 510 in most cases with minimal corrosion risk. Within these constraints, more heat can potentially be extracted from recycled flue gas 454 in exchanger 510 compared with the heat extracted from the same amount of flue gas if it were directed instead to the gas-to-gas air preheater 502. A methodology for estimating acid dew points in combustion flue gasses is given in: A. G. Okkes, “Get acid dew point of flue gas,” Hydrocarbon Processing, July, 1987, pp. 53-55.

The entire contents of each and every patent and non-patent publication cited herein are hereby incorporated by reference, except that in the event of any inconsistent disclosure or definition from the present specification, the disclosure or definition herein shall be deemed to prevail.

The foregoing detailed description and the accompanying drawings have been provided by way of explanation and illustration, and are not intended to limit the scope of the appended claims. Many variations in the presently preferred embodiments illustrated herein will be apparent to one of ordinary skill in the art, and remain within the scope of the appended claims and their equivalents.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims can, alternatively, be made to depend in the alternative from any preceding claim—whether independent or dependent—and that such new combinations are to be understood as forming a part of the present specification.

1. A process for manufacturing a purified aromatic carboxylic acid comprising:
generating high-pressure steam from boiler feed water supplied to a boiler, the boiler producing a flue gas;
removing a portion of the flue gas from the boiler and pre-heating the makeup water with removed flue gas;
pre-heating at least a portion of the boiler feed water prior to its introduction into the boiler with a first portion of the high-pressure steam;
heating a crude aromatic carboxylic acid in a heating zone with a second portion of the high-pressure steam, whereby the high pressure steam is condensed in the heating zone to form a high-pressure condensate; and
purifying the crude aromatic carboxylic acid to form a purified aromatic carboxylic acid;
wherein the boiler feed water comprises at least a portion of the high-pressure condensate.

2. The process of claim 1, wherein the boiler feed water further comprises make-up water.

3. The process of claim 2, wherein pre-heating at least a portion of the boiler feed water comprises pre-heating the make-up water.

4. The process of claim 3, wherein the boiler feed water further comprises at least a portion of the first portion of the high pressure steam.

5. The process of claim 3, wherein pre-heating the make-up water comprises exchanging heat with at least a portion of the first portion of the high pressure steam to form a second condensate from the high pressure steam, the boiler feed water further comprising the second condensate.

6. The process of claim 5, further comprising mixing the high pressure condensate, the second condensate, and the make-up water in a high pressure deaerator to form the boiler feed water.

7. The process of claim 6, wherein mixing the high pressure condensate, the second condensate, and the make-up water in the high pressure deaerator to form the boiler feed water further comprises mixing at least a portion of the first portion of high pressure steam.

8. The process of claim 1, wherein the makeup water is pre-heated with flue gas upstream of heating the makeup water with a portion of the high pressure steam.

9. The process of claim 1 wherein the aromatic carboxylic acid comprises terephthalic acid.

10. The process of claim 1, wherein the portion of flue gas removed from the boiler is 50% or less of the volume of flue gas produced in the boiler.

11. The process of claim 1, wherein the portion of flue gas removed from the boiler is 30% or less of the volume of flue gas produced in the boiler.

12. The process of claim 1, wherein the portion of flue gas removed from the boiler is 20% or less of the volume of flue gas produced in the boiler.

13. The process of claim 1, further comprising feeding a source of oxygen and a fuel to the boiler to support combustion.

14. The process of claim 13, wherein at least a portion of the flue gas used to pre-heat the boiler feed water is added to the source of oxygen prior to the introduction of the source of oxygen to the boiler.

15. The process of claim 13, wherein the source of oxygen is pre-heated prior to its introduction to the boiler by at least a portion of the flue gas remaining after removal of the portion of flue gas used to pre-heat the boiler feed water.
